(19) United States
(12) Patent Application Publication
(10) Pub. No.: US 2025/0371123 A1
(43) Pub. Date: Dec. 4, 2025

(54) Dynamic Token Generation On Eyesight Display With Photoplethysmography

(71) Applicant: Apple Inc., Cupertino, CA (US)

(72) Inventors: Arun Srivatsan Rangaprasad, Sunnyvale, CA (US); Anselm Grundhoefer, Campbell, CA (US); Jay C Mung, San Jose, CA (US)

(21) Appl. No.: 19/198,397

(22) Filed: May 5, 2025

Related U.S. Application Data
(60) Provisional application No. 63/654,200, filed on May 31, 2024.

(51) Int. Cl. G06F 21/32; A61B 5/00; A61B 5/024; A61B 5/1171; G06F 3/01

(52) U.S. Cl. CPC G06F 21/32; A61B 5/1176; A61B 5/6803; A61B 5/7257; G06F 3/015; A61B 5/02427

(57) ABSTRACT

Various implementations disclosed herein include devices, systems, and methods that embed information in video presented on an outward display of a wearable device. For example, a process may include generating a video signal depicting a current appearance of a face portion. Changes in an attribute of the face portion in the video signal over time may correspond to a current heart rate of a user wearing the wearable electronic device. The process may further include embedding data into the video signal by altering the attribute of the face portion in the video signal over time such that the changes in the attribute of the face portion in the video signal over time correspond to both the current heart rate and the data. The process may further include presenting the video signal depicting the current appearance of the face portion on an outward-facing display of the wearable electronic device.

### CROSS-REFERENCE TO RELATED APPLICATIONS

This application claims the benefit of U.S. Provisional Application Ser. No. 63/654,200 filed May 31, 2024, which is incorporated herein in its entirety.

### TECHNICAL FIELD

The present disclosure generally relates to electronic devices, and in particular, to systems, methods, and devices for sharing an encoded message embedded within a video.

### BACKGROUND

Existing techniques for sharing data between devices may be improved with respect to accuracy and security to provide discrete data sharing functionality.

### SUMMARY

Various implementations disclosed herein include devices, systems, and methods that embed information (e.g., an alpha numeric code) within a video presented via an outward display of a head mounted device (HMD) thereby enabling a different device to capture images of the HMD while the video is being displayed. In some implementations, the captured images may be used to identify the information. The information may be discretely and securely transferred between devices (e.g., the HMD and a receiving (image capture device) via the embedded code (e.g., a token).

In some implementations, video being displayed via the outward display of the HMD may be configured to display a portion (e.g., an eye region) of a face of a user of the HMD. In some implementations, a heartrate of the user may be extracted from the displayed portion (e.g., a patch of skin) of the user's face and the information may be embedded within the video based on the heartrate. In some implementations, determining the heartrate may involve using remote photoplethysmography (rPPG) to extract an average intensity over a displayed portion of the user's face to produce a raw signal to be filtered and brought into the frequency domain (via a Fast Fourier Transform (FFT)) to illustrate a peak with respect to the heartrate. The code may be embedded within the video by adding additional, discretized peaks into the signal. For example, peaks of 2-3 different peak heights scaled according to the heartrate peak height may be added into the signal.

In some implementations, the receiving device may use rPPG to determine a heartrate of the user (of the HMD) and interpret image data of the HMD to, inter alia, extract the embedded information. For example, the receiving device may identify a patch of the HMD user's skin and a patch of skin displayed via the HMD user's device. In some implementations, the heartrate may be identified from each the skin patches and matched to authenticate the user to, inter alia, confirm that the user is currently wearing the HMD. Subsequently the embedded information may be identified. In some implementations, additional heartrate information (e.g., from other devices worn by the user) may be used to further enhance user authentication techniques. The other devices may include, inter alia, a smart watch, a tablet computer, wireless headphones, a mobile phone, etc. The embedded code may additionally be used to automatically unlock device to device communications, initiate sharing between the devices, authenticate the user, etc.

In some implementations, wearable device has a processor (e.g., one or more processors) that executes instructions stored in a non-transitory computer-readable medium to perform a method. The method performs one or more steps or processes. In some implementations, the wearable device generates a video signal depicting a current appearance of a face portion. The video signal may be generated based on sensor data captured via one or more sensors. In some implementations, changes in an attribute of the face portion in the video signal over time may correspond to a current heart rate of a user wearing the wearable electronic device. The method may further embed data into the video signal by altering the attribute of the face portion in the video signal over time such that the changes in the attribute of the face portion in the video signal over time correspond to both the current heart rate and the data. The method may further present the video signal depicting the current appearance of the face portion on an outward-facing display of the wearable electronic device.

In accordance with some implementations, a device includes one or more processors, a non-transitory memory, and one or more programs; the one or more programs are stored in the non-transitory memory and configured to be executed by the one or more processors and the one or more programs include instructions for performing or causing performance of any of the methods described herein. In accordance with some implementations, a non-transitory computer readable storage medium has stored therein instructions, which, when executed by one or more processors of a device, cause the device to perform or cause performance of any of the methods described herein. In accordance with some implementations, a device includes: one or more processors, a non-transitory memory, and means for performing or causing performance of any of the methods described herein.

### BRIEF DESCRIPTION OF THE DRAWINGS

So that the present disclosure can be understood by those of ordinary skill in the art, a more detailed description may be had by reference to aspects of some illustrative implementations, some of which are shown in the accompanying drawings.

FIG. 1 illustrates an environment with a device presenting a view of a face portion of a user, in accordance with some implementations.

FIG. 2 illustrates an enlarged visualization of the head of the user and the device of FIG. 1, in accordance with some implementations.

FIG. 3 is a process flow chart illustrating an exemplary rendering technique, in accordance with some implementations.

FIG. 4 illustrates a process for determining a user heartrate estimate of a user wearing a wearable device, in accordance with some implementations.

FIG. 5 illustrates a view of a process for identifying a heartrate of a user to decode data embedded in a video stream, in accordance with some implementations.

FIG. 6 illustrates an alternative view of a process for identifying a heartrate of a user to decode data embedded in a video stream, in accordance with some implementations.

FIG. 7 is a flowchart representation of an exemplary method for sharing an encoded message embedded within a video, in accordance with some implementations.

FIG. 8 is a block diagram illustrating device components of an exemplary device according to some implementations.

In accordance with common practice the various features illustrated in the drawings may not be drawn to scale. Accordingly, the dimensions of the various features may be arbitrarily expanded or reduced for clarity. In addition, some of the drawings may not depict all of the components of a given system, method or device. Finally, like reference numerals may be used to denote like features throughout the specification and figures.

### DESCRIPTION

Numerous details are described in order to provide a thorough understanding of the example implementations shown in the drawings. However, the drawings merely show some example aspects of the present disclosure and are therefore not to be considered limiting. Those of ordinary skill in the art will appreciate that other effective aspects or variants do not include all of the specific details described herein. Moreover, well-known systems, methods, components, devices and circuits have not been described in exhaustive detail so as not to obscure more pertinent aspects of the example implementations described herein.

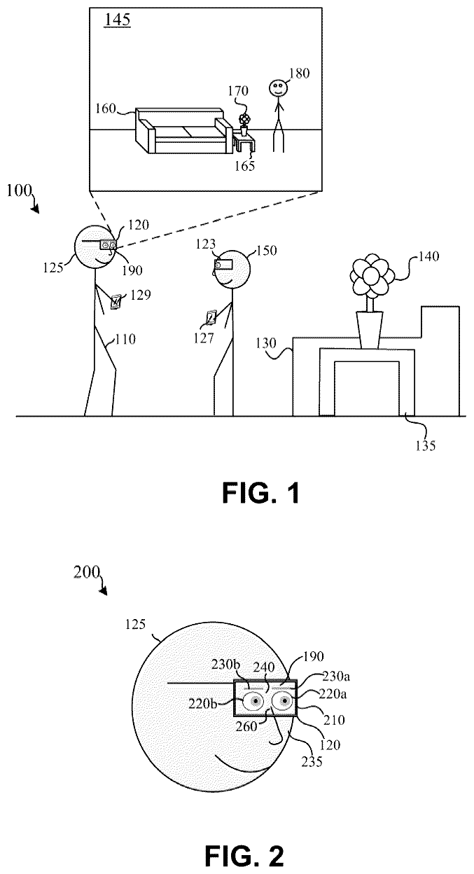

FIG. 1 illustrates an example physical environment 100 (e.g., a room) including a device 120, a device 123, a device 127, and a device 129. In some implementations, the device 120 displays content to a user 110, e.g., extended reality (XR) content. For example, content may include representations of the physical environment 100 (e.g., passthrough video) and/or virtual content, e.g., user interface elements such as menus, buttons, icons, text boxes, graphics, avatars of another device user, etc. In the example of FIG. 1, the environment 100 includes another person 150 with device 123 and/or device 127, a couch 130, a table 135, and flowers 140, and the device 120 displays a view 145 to user 110 on one or more internal displays. The view 145 includes a depiction 160 of the couch 130, a depiction 165 of the table 135, a depiction 170 of the flowers 140, and a depiction 180 of the other person 150.

In some implementations, the device 120 includes virtual content (not shown) in the view 145. Such virtual content may include a graphical user interface (GUI). In some implementations, the user 120 interacts with such virtual content through virtual finger contacts, hand gestures, voice commands, use of an input device, and/or other input mechanisms. In some implementations, the virtual content enables one or more application functions including, but not limited to, image editing, drawing, presenting, word processing, website creating, disk authoring, spreadsheet making, game playing, telephoning, video conferencing, e-mailing, instant messaging, workout support, digital photographing, digital videoing, web browsing, digital music playing, and/or digital video playing. Executable instructions for performing these functions may be included in a computer readable storage medium or other computer program products configured for execution by one or more processors.

While this example and other examples discussed herein illustrate a single device 120 in a real-world environment 100, the techniques disclosed herein are applicable to multiple devices performing some or all of the functions. In some implementations, the device 120 is a wearable device such as an XR headset, smart-glasses, or other HMD, as illustrated in FIG. 1. In some implementations, the device 120 is a handheld electronic device (e.g., a smartphone or a tablet) held or otherwise positioned in front of the user's face. In some implementations the device 120 is a laptop computer or a desktop computer held or otherwise positioned in front of the user's face. In some implementations, device 123, device 127, and/or device 129 may be, inter alia, a smart watch, a tablet computer, wireless headphones, a mobile phone, an HMD, etc.

The device 120 obtains image data, depth data, motion data, and/or other sensor data associated with the user 110 and/or the physical environment 100 via one or more sensors. For example, the device 120 may obtain infrared (IR) images of a portion of the user's head 125 from one or more inward-facing infrared cameras while the device 120 is being worn by the user 110. In some implementations, the sensors may include any number of sensors that acquire data relevant to the appearance of the user 110. For example, when wearing an HMD, one or more sensors (e.g., cameras inside the HMD) may acquire images associated with the eyes and surrounding areas of the user and one or more sensors on the outside of the device 120 may acquire images associated with the user's body (e.g., hands, lower face, forehead, shoulders, torso, feet, etc.) and/or the physical environment 100.

In some implementations, the device 120 includes an eye imaging and/or eye tracking system for detecting eye position and eye movements via eye gaze characteristic data. For example, an eye tracking system may include one or more infrared (IR) light-emitting diodes (LEDs), an eye tracking camera (e.g., near-IR (NIR) camera), and an illumination source (e.g., an NIR light source) that emits light (e.g., NIR light) towards the eyes of the user 25. Moreover, the illumination source of the device 120 may emit NIR light to illuminate the eyes of the user 110 and the NIR camera may capture images of the eyes of the user 110. In some implementations, images captured by the eye tracking system may be analyzed to detect position and movements of the eyes of the user 110, or to detect other information about the eyes such as appearance, shape, state (e.g., wide open, squinting, etc.), pupil dilation, or pupil diameter. Moreover, the point of gaze estimated from the eye tracking images may enable gaze-based interaction with content shown on one or more near-eye displays of the device 120.

In some implementations, the device 120 includes a hand tracking system for detecting hand position, hand gestures/configurations, and hand movements via hand tracking data. For example, the device 120 may include one or more outward facing cameras, depth sensors, or other sensors that capture sensor data from which a user skeleton can be generated and used to track the user's hands. Hand tracking information, e.g., gestures, and/or gaze tracking data may be used to provide input to the device 120.

The device 120 uses sensor data (e.g., live and/or previously-captured) to present a view 190 depicting a video of a face portion (e.g., an eye region) of the user 110 that would otherwise be blocked by the device 120. The view 190 is presented on an outward facing display of the user's device 120 and may be visible to the other person 150. The other person 150 may observe the view depicting the face portion of user 110 to see a relatively accurate representation of the current and moving face portion of the user 110. Likewise, device 123 or 127 may capture images of device 120 while it is displaying the video and use the images to identify information (in the video) such as an embedded code. Accordingly, the information may be discretely and securely transferred from device 120 to device 123 and/or device 127. The information captured by device 123 and/or device 127 may be used to extract a user's heartrate for authentication as described, infra. The view may be aligned to provide 3D accuracy, e.g., such that the other person 150 sees the face portion of the user 110 with face portion appearing in its actual 3D position, e.g., as if a front area of the device 120 were transparent and the other person were viewing the face of the user 110 directly through the transparent area.

FIG. 2 provides an enlarged illustration of the head of the user 110 and the device 120 of FIG. 1. As illustrated, the device 120 includes an outward-facing display 210 (e.g., on the front surface of device 120 and facing outward away from the eyes of the user 110 to display content (e.g., a video signal that includes an embedded code) to one or more other persons via a device (e.g., device 123 and/or device 127 of FIG. 1) in the physical environment 100). In some implementations, the display 210 is only activated to display content (e.g., the user's face portion) when one or more other persons are detected within the physical environment 100, detected within a particular distance or area, detected to be looking at the device 120, or based on other suitable criteria.

The display 210 presents view 190, which in this example includes a depiction 220a of a left eye of the user 110, a depiction 220b of a right eye of the user 110, a depiction 230a of the left eyebrow of the user 110, a depiction 230b of the right eyebrow of the user 110, depiction 240 of skin (e.g., a patch of skin for enabling heartrate detection) around/near the eyes of the user 110, and depiction 260 of an upper nose portion of the user 110, etc. The view 190 provides depictions of a face portion that would otherwise be blocked from view by the device 120. The display of the user's face portion may be configured to enable observers (e.g., the other person 150 and associated devices 123 and 127) to see the user's current eyes and facial expressions as if the person 150 were seeing through a clear device at the actual eyes and facial expressions of user 110.

The view 190 may be updated over time, for example, providing a live view of the appearance of the face portion of the user such that the person 150 sees the eyes and facial appearance/expressions of the user 110 changing over time. Accordingly, such a live updated view 190 may be based on live updated sensor data, e.g., capturing inner camera data signal over time and repeatedly updating the representation of the face portion for each point in time, e.g., every frame, every 5 frames, every 10 frames of the display cycle.

The view 190 of the user's face portion may be configured to be realistic and correspond to the user's current appearance. This may be achieved or facilitated, for example, by utilizing both live and previously-captured information about the appearance of the user's face portion. In one example, enrollment data (e.g., from an enrollment period prior to the live experience) and live data are combined to provide a view of the user's face portion. The live data may provide information about the current state of the face portion while the enrollment data may provide information about one or more attributes of the face portion that are un-attenable or not captured as well in the live environment (e.g., corresponding to portions of the face portion that are blocked from live sensor capture by the device being worn or corresponding to color, 3D shape, or other elements of the face portion that are not captured or depicted as accurately by the live sensors). In one example, prior enrollment data is captured while the user 110 is not wearing the device 120 while the live data is captured while the user 110 is wearing the device 120. Some implementations combine live data, e.g., based on live eye camera data, with enrollment data, e.g., enrolled panels based on views of the face without the face being blocked by the device and in one or more lighting conditions.

The view 190 of the user's face portion may be configured to present the face portion with 3D spatial accuracy, e.g., each eye appearing to be in its actual 3D position for different observation viewpoints around the user. This may involve determining a 3D appearance of the face portion (e.g., mapping an image of the face portion onto a 3D model of the face portion) and providing a view of the 3D appearance of the face portion for a particular observer viewpoint/direction, e.g., based on the relative positioning of the other person 150. The view may be provided based on mapping combined data (e.g., an inferred image/panel representing the current appearance of the user's face portion based on live and previously-captured enrollment data) to a 3D mesh and then providing the view of the 3D mesh (on the external display) based on an observer viewpoint so that the eyes appear to an observer at that viewpoint in their actual 3D position. The shape of the display 210 and/or its position relative to the user 110 (e.g., where it is on the user's face) may be used in providing the view so that the eyes and surrounding areas appear to be spatially accurate.

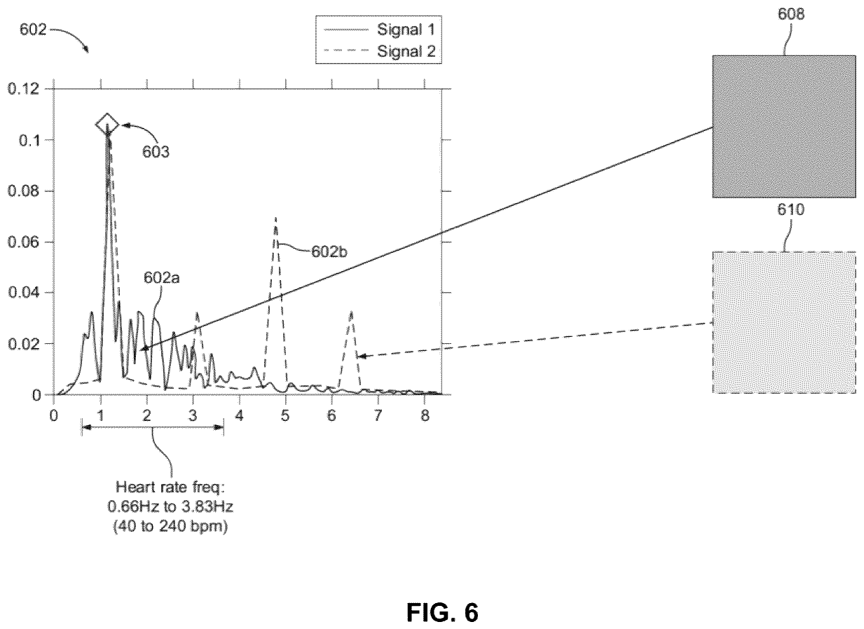

In some implementations, a biometric token (e.g., comprising a code/information embedded in a video) may be generated for sharing between devices (e.g., sharing between device 120 and 123 or 127) via usage of view 190 such that when view 190 is being displayed, an intensity of for example, depiction 240 of skin (of user 110) may be modulated to encode information within a frequency spectrum. Accordingly, when another user (e.g., user 150) scans (e.g., via device 123 or 127) view 190 of user 110, an associated image sensor (e.g., a camera) may extract a displayed view of skin patches from the user's head 125 (e.g., displayed skin patch view 608 of FIG. 6 as described, infra) and from display 210 (e.g., displayed skin patch view 610 of FIG. 6 as described, infra) providing depictions 220a and 220b of eyes and a depiction 240 of skin around/near the eyes and surrounding areas. The displayed view of the skin patches from the user's head 125 may be used to determine a heartrate of the user for generating the biometric token. In some implementations, determining the heartrate of the user may involve using remote rPPG techniques by extracting an average intensity over a patch to produce a raw signal that is filtered and brought into the frequency domain (via FFT) to illustrate a peak of the heartrate. For example, the token/code may be embedded by adding additional discretized peaks into the raw signal (e.g., by adding peaks of 2-3 different peak heights that are scaled according to the heartrate peak height). In response, a receiving device (e.g., device 123 and device 127 of FIG. 1) may use rPPG to determine the user's heartrate and interpret image data (e.g., of view 190) of the associated device accordingly to extract the embedded information. For example, the receiving device may be configured to identify a patch of the user's skin and a patch of skin displayed within view 190. In response, the heartrate may be identified from each the skin patches and matched to authenticate the user (e.g., confirm that the user is wearing the device live) and identify the embedded information.

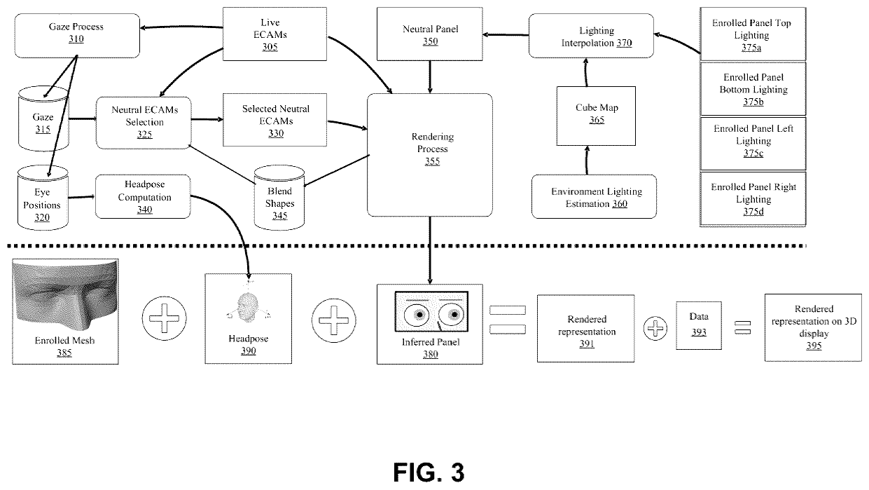

FIG. 3 is a process flow chart illustrating an exemplary rendering technique. In this example, a rendering process 355 receives various inputs from both live and previously-captured sources and outputs a representation of a face portion of the user, i.e., inferred panel 380. The rendering process 355 may be implemented as an algorithm or a machine learning model such as a neural network that is trained to produce an inferred panel or other such representation based on the combined inputs. Such a network may use training data that provides accurate depictions of current face portions corresponding to training input data, e.g., actual or synthetically-generated renderings of the training face portions mimicking sensor captured-data.

In the example of FIG. 3, the rendering process 355 receives input that includes a neutral panel 350 generated based (at least in part) on previously-captured user data, e.g., sensor data from a previously-completed enrollment process in which images and/or other sensor data of the user were captured. Such images may correspond to different lighting conditions, different viewpoints, and/or different facial expressions, e.g., one or more images captured with light illuminating the user from the right side, one or more images captured with light illuminating the user from the left side, one or more images captured with light illuminating the user from the top, one or more images captured with light illuminating the user from below the user's face, one or more images captured with the user's face turned to the left, one or more images captured with the user's face turned to the right, one or more images captured with the user's face tilted up, one or more images captured with the user's face tilted down, one or more images captured with the user's face smiling, one or more images captured with the user's face exhibiting a neutral expression, one or more images captured with the user's face exhibiting an specific facial expression, one or more images captured with the user's mouth open, one or more images captured with the user's mouth closed, one or more images captured with the user's eyes open, one or more images captured with the user's eyes closed, one or more images captured with the user's eye brows raised, one or more images captured with the user's eye brows down, etc.

In some implementations, during an enrollment process (on the same or different device), the user is guided to capture enrollment sensor data. For example, the user may be guided to capture images of themselves by holding the device out in front of them such that sensors that would normally be outward facing when the device is being worn would be oriented towards the user's face. Such outward facing sensors may capture data of a type or quality that inward facing sensors on the device do not. For example, inward-facing sensors on the device may be IR cameras while the outward facing sensors may capture color image data not captured by the IR cameras. Sensor data captured during enrollment may also be captured while the user is not wearing the device and thus include or represent parts of the user's face that are blocked (from capture by any sensor) while the device is being worn, e.g., parts of the user's face that are covered or in contact with a light seal of an HMD device while the HMD is being worn.

In some implementations, enrollment data comprises data that is generated based on captured sensor data. For example, images of the user may be captured during an enrollment process which occurred in a particular lighting condition (e.g., light from the top). This data may be used to generate enrolled panels corresponding to different lighting conditions, e.g., enrolled panel top lighting 375a depicting a portion of the user's face illuminated by top lighting, enrolled panel bottom lighting 375b depicting the portion of the user's face illuminated by bottom lighting, enrolled panel left lighting 375c depicting the portion of the user's face illuminated by left lighting, and enrolled panel right lighting 375d depicting a portion of the user's face illuminated by right lighting. In this example, these enrolled panels 375a-d are orthographic projections of a portion of the user's face generated based on the sensor data obtained at enrollment to which synthetic lighting has been included.

In FIG. 3, at runtime/rendering time, an environment lighting estimation 360 is performed by the device, e.g., determining the locations of one or more light sources in the environment and/or the directions relative to the device/user of light in the environment. In this example, the lighting estimation is used to provide a cube map 365 representing the lighting which is used at lighting interpolation block 370 to generate a neutral panel (e.g., corresponding to the current lighting condition represented by the cube map 365 with the user's face in a neutral configuration, i.e., eyes open, looking straight forward, neutral expression, etc.). This may involve interpolating values from the enrolled panels 375a-d. For example, if the face is being lit from the bottom left side, then the neutral panel may be generated by interpolating between the enrolled panel lift lighting 375c and the enrolled panel bottom lighting 375b. The amount of blending or other interpolation may be based on the specific location and characteristics of a light source and/or amount of light illuminating the face from a particular direction.

The rendering process 355 uses the neutral panel as one of its inputs in producing the inferred panel 380.

In FIG. 3, the rendering process 355 also uses eye camera data which may be based at least in part on live sensor data, e.g., sensor data being currently captured during the user's wearing of the device and the presentation of a view of the face portion on an external display of the device. In this example, live ECAMS (i.e., eye cameras) capture sensor data (e.g., IR images) of parts of the user's face that are inside and not covered by the device while the device is being worn by the user. Such parts of the user face may, but do not necessarily, include the user's eyes, eye lids, eyebrows, and/or surrounding facial areas but do not include areas of the face that are covered by portions of the device contacting the user's face (e.g., the device's light seal). Live ECAM data may be captured by the live ECAMS 305 for multiple purposes, e.g., for use in tracking the user's gaze for input and/or other purposes as well as for generating a view of the user's face portion for display on an external display of the device. Using the same eye region sensors for multiple purposes may improve device efficiency and enhanced performance properties.

In the example of FIG. 3, the live ECAMs 305 provide sensor data (e.g., IR images of each of the eyes and surrounding areas) to the rendering process 355 as well as to a gaze process 310 and a neutral ECAMs selection 325 block. The gaze process 310 uses the data from the live ECAMS 305 to determine eye characteristics such as gaze 315 (e.g., gaze direction) and/or eye positions 320 (e.g., 6DOF eye ball poses). The gaze 315 is used by neutral ECAMs selection 325 block, along with the data from the live ECAMS 305, to produce selected neutral ECAMs 330, which provide data e.g., image data corresponding to neutral eye state in which the eye is open and looking straight forward.

The rendering process 355 may produce inferred panel 380 and/or blendshapes. Blendshapes may represent facial features and/or expressions. In one example, blendshapes represent a detected facial expression. In one example, blendshapes use a dictionary of named coefficients representing the detected facial expression in terms of the movement of specific facial features. The neutral ECAMs selection 325 block may use gaze 315 and/or the blendshapes 345 to compute information such as a neutral score. In some implementations, at each frame, the neutral ECAMs are replaced by the live ECAMS 305 each time the neutral score is improved.

The live ECAMs 305 data and the selected neutral ECAMs 330 data is used by the rendering process 355 in producing the inferred panel 380. In this example, the rendering process receives input including the neutral panel 350, live ECAMs 305 data, and selected neutral ECAMs 330 data, and produces an inferred panel 380 as output. In some implementations, the live ECAMs 305 data and selected neutral ECAMs 330 data is compared to estimate a difference, e.g., how much and/or how features in the live ECAMs 305 data differ from the same features in the selected neutral ECAMs 330 data. This may involve identifying such features in corresponding eye images from each set of data and determining amounts of movement/difference between their locations. In some implementations, the rendering process 355 is a neural network or other machine learning model that accounts for such differences (e.g., implicitly without necessarily being explicitly trained to do so) in modifying the input neutral panel 350 data to produce inferred panel 380.

Conceptually, the rendering process can use the live ECAMs 305 data to determine how much and how the current eye area appearance differs from its neutral appearance and then apply the determined difference to modify the neutral panel 350 to produce an inferred panel 380 correspond to the current eye area appearance. In this way, in this example, previously-captured face portion attributes (e.g., from enrollment) that are present/represented in the neutral panel 350 are combined with live data from the live ECAMs 305 to produce an inferred panel 380 that corresponds to the current appearance of the user's face portion while also including accurate attributes from the previously obtained (e.g., enrollment) data.

In the lower portion of FIG. 3, the inferred panel 380 produced by the rendering process 355 is combined with other data to produce a rendered representation 391. In this example, the inferred panel 380 is applied to add color/texture to an enrolled mesh 385 (e.g., a 3D model of the face portion generated previously such as during the user's enrollment while the user was not wearing the device).

Headpose 390 information may also be determined, for example, by headpose computation 340 block using eye position data and/or other data such as IMU data, SLAM data, VIO data, etc. to determine a current headpose 390. Such a headpose may identify position and/or orientation attributes of the device/user's head, e.g., identifying a 6DOF pose of the user's head. Headpose 390 may be used to determine where to spatially position the textured 3D mesh (combination of enrolled mesh 385 with inferred panel 380) in relation to the user's head/device 3D position for rendering purposes, e.g., where the face portion is positioned in a 3D space relative to a viewpoint position/direction for rendering purposes.

In some implementations, changes in an attribute (e.g., intensity) of a face portion (of inferred panel 380) of rendered representation 391 (e.g., a video signal) over time may correspond to a current heart rate of the user. For example, rendered representation 391 may include an intensity based on live ECAMs data 305.

In some implementations, data 393 (e.g., a numerical code) may be embedded into rendered representation 391 by altering the attribute of the face portion in the rendered representation 391 over time such that the changes in the attribute of the face portion in the rendered representation 391 over time correspond to the current heart rate and the data 393.

In some implementations, the rendered representation 391 depicting the current appearance of the face portion may be presented as a rendered representation on a 3D display 395 (on an outward-facing display of a wearable electronic device).

A 3D position or viewpoint direction of an observer may be estimated and used in producing the rendered representation (of the face portion) on the 3D display 395. An observer may see an image of the face portion displayed on an external 2D display of the device, e.g., on a flat or curved-flat front surface such that each of the displayed eyes and other areas of the face portion appear to be at locations at which they would appear if the device were see through and the observer was observing the user's actual face.

In some implementations, the display provides different views for different observer viewpoints, e.g., using a lenticular display that displays images (e.g., 10+, 15+, 25+, etc. images) for different observer viewpoints such that, from a given viewpoint an observer, views an appropriate view, e.g., with the displayed face portion's 3D position appearing to match the corresponding actual face portion's actual current position. In such a configuration, an observer's actual viewpoint need not be determined since the observer will view an appropriate image for their current viewpoint based on the characteristics of the display device.

The rendering process of FIG. 3 can be repeated over time, for example, such that an observer sees what appears to be a live 3D video of the user's face portion including eye movements and facial expression changing over time on an external display of the device.

In one example, the live ECAMs 305 data is received as a series of frames and the rendering process 355 produces an inferred panel 380 that is used to display an updated rendered representation (of the face portion) on the 3D display 395 for each eye data frame. In other implementations, the rendered representation on the 3D display 395 is updated less frequently, e.g., every other eye data frame, every 10th eye data frame, etc.

Some of the data in the process need not be updated during the live rendering. For example, the same set of enrolled panels 375a-d may be used for multiple frames, e.g., for all frames, during the live rendering of the face portion. In this example, the lighting interpolation 370 may use that static data (i.e., enrolled panels 375a-d) based on current environment lighting estimation 360 that may or may not be updated during the live rendering. In one example, the environment lighting estimation 360 and lighting interpolation 370 occur just once at the beginning of a user experience. In another example, the lighting estimation 360 and lighting interpolation 370 occurs during every frame of data capture during a user experience. In other examples, these processes occur periodically and/or based on detecting conditions (e.g., lighting) changing above a threshold during a user experience.

The enrolled mesh 385 similarly need not be updated during the live rendering. The same enrolled mesh 385 may be used for all rendered representations 395 during a user experience. In another implementation, an enrolled mesh 385 is updated during the user experience, e.g., via an algorithm or machine learning process, that uses live data to modify an enrolled mesh 385 before applying the current inferred panel 380.

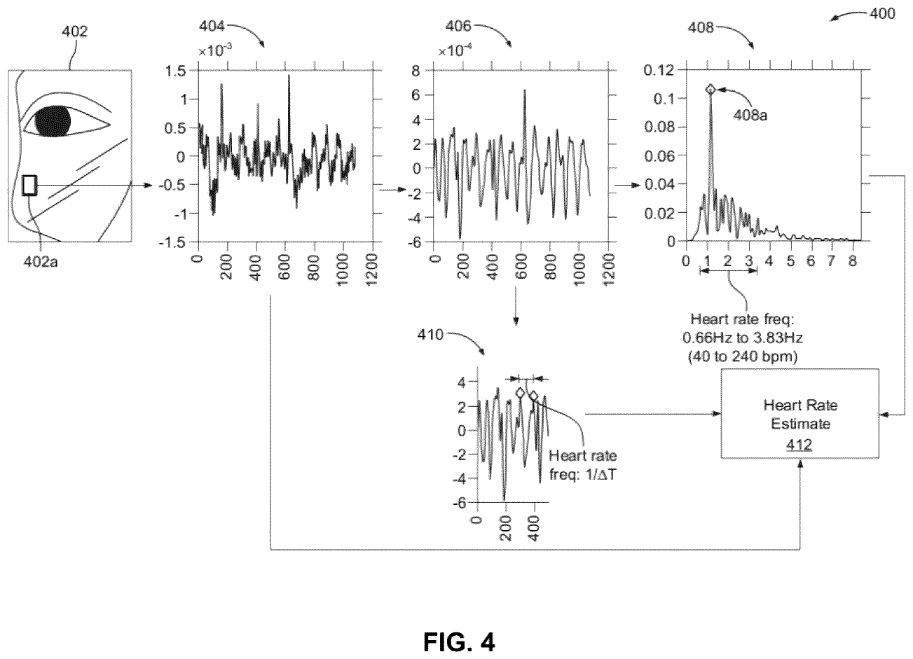

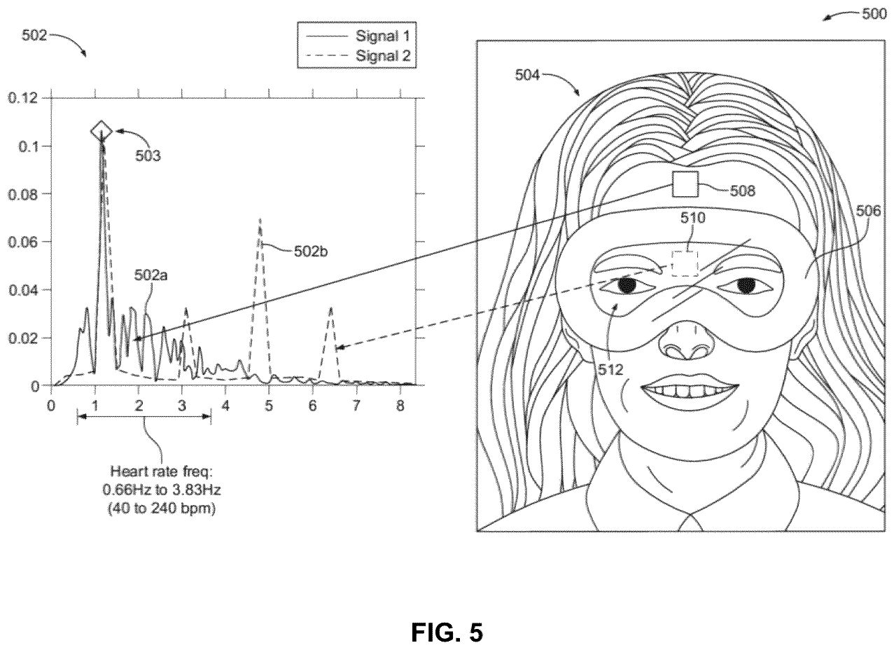

FIG. 4 illustrates a process 400 for determining a user heartrate estimate 412 of a user wearing a wearable device such as an HMD. In some implementations, a user heartrate may be extracted by analyzing an ECAM feed (e.g., of live ECAMs 305 of FIG. 3) displaying a portion 402 (e.g., eye region) of a user's face from which the user's heartrate may be extracted. Subsequently, a remote rPPG process may be executed with respect to band pass and noise filtering operations to extract an average intensity over a patch 402a of skin of portion 402 of the user's face to generate a raw intensity signal 404 that may be used to directly determine user heartrate estimate 412. Alternatively, the raw intensity signal 404 may be filtered to generate a noise filtered and band pass signal 406 to be brought into a frequency domain via a Fast Fourier Transform (FFT) 408 to illustrate a peak 408a of the heartrate at a specified frequency representing an overall heart rate. Subsequently, the specified frequency may be converted into heart rate beats per minute (bpm) to determine user heartrate estimate 412. As a further alternative, user heartrate estimate 412 may be determined by inputting noise filtered and band pass signal 406 into a neural network (NN) 410 that generates as an output, user heartrate estimate 412. In some implementations, data (e.g., a numerical code) may be embedded into a video signal by altering the average intensity over patch 402a in the video signal over time such that the changes in the attribute of portion 402 of the user in the video signal over time to correspond to user heartrate estimate 412 and the data.

FIG. 5 illustrates a view 500 of a process for identifying a heartrate of a user 504 to decode data embedded in a video stream and/or authenticate user 604 for discrete transfer/sharing of the data from a wearable device 506 to a receiving device. The embedded data (e.g., a 3-4 digit code) in the video stream may be presented on an outward display 512 of wearable device 506 so that a reading device (not shown) may capture images of wearable device 506 while it is displaying the video stream. The images may be used to identify the data. For example, a patch 508 of skin of user 504 directly visible in the images may be identified. Likewise, a patch 510 of skin of user 504 displayed via outward facing display 512 of wearable device 506 may be identified. Subsequently, a heartrate (of user 504) identified in patch 508 and corresponding to a signal 502a is compared to a heartrate (of user 504) identified in patch 510 and corresponding to a signal 502b and in response, the data is decoded and/or user 504 is identified based on results of the heartrate comparison. For example, an intensity of signals 502a and 502b may be brought into a frequency domain to determine a match to a tallest peak 503. Likewise, additional peaks of signals 502a and 502b may correspond to data values and the additional peaks may further be associated with height values corresponding to discrete data values. In some implementations, the height values of the additional peaks may be dependent upon a height of a peak corresponding to the heartrate.

FIG. 6 illustrates an alternative view 600 of a process for identifying a heartrate of a user to decode data embedded in a video stream and/or authenticating the user for discrete transfer/sharing of the data from a wearable device to a receiving device. In contrast with view 500 of FIG. 5, view 600 of FIG. 6 illustrates a first heartrate signal 608 of the user identified in via a device 608 (e.g., device 129 of FIG. 1 such as a smartwatch, wireless headphones, etc.) and a second heartrate signal 610 of the user identified in via a device 710 (e.g., device 120 of FIG. 1 such as, inter alia, an HMD). Device 608 may identify heartrate signal 702a via usage of, inter alia, photoplethysmography (PPG), an inertial measurement unit (IMU), etc. Likewise, device 610 may identify heartrate signal 602b via usage of a patch of skin of the user displayed via an outward facing display of a wearable device as described with respect FIG. 5, supra. Subsequently, a heartrate of the user identified via device 608 and corresponding to signal 602a is compared a heartrate of the user identified via device 610 and corresponding to signal 602b and in response, the data of the wearable device is decoded and/or the user is identified based on results of the heartrate comparison for the two devices 608 and 610. For example, an intensity of signals 602a and 602b may be brought into a frequency domain to determine a match for a tallest peak 603. Likewise, additional peaks of signals 602a and 602b may correspond to data values and the additional peaks may further be associated with height values corresponding to discrete data values. In some implementations, the height values of the additional peaks may be dependent upon a height of a peak corresponding to the heartrate. Accordingly, device 608 and device 610 are utilized in combination to enable code to be scanned via a receiving device.

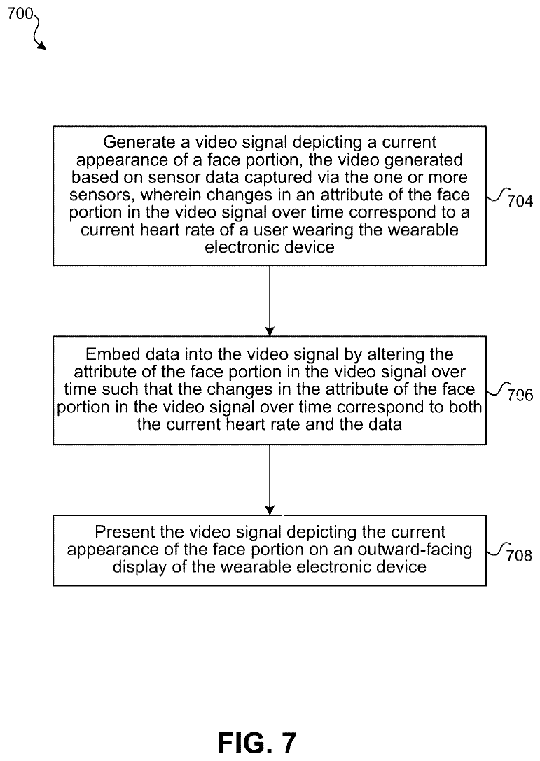

FIG. 7 is a flowchart representation of an exemplary method 700 for sharing an encoded message embedded within a video, in accordance with some implementations. In some implementations, the method 700 is performed by a device, such as a mobile device, desktop, laptop, HMD, or server device. In some implementations, the device has a screen for displaying images and/or a screen for viewing stereoscopic images such as a head-mounted display (HMD such as e.g., device 120, 123, 127 or 129 of FIG. 1). In some implementations, the method 700 is performed by processing logic, including hardware, firmware, software, or a combination thereof. In some implementations, the method 700 is performed by a processor executing code stored in a non-transitory computer-readable medium (e.g., a memory). Each of the blocks in the method 700 may be enabled and executed in any order.

At block 704, the method 700 generates a video signal depicting a current appearance of a face portion of a user wearing a wearable device (e.g., device 120 of FIG. 1) such as an HMD. The video signal generated based on sensor data captured via one or more sensors. In some implementations, changes in an attribute, such as intensity, of the face portion in the video signal over time may correspond to a current heart rate of a user wearing a wearable electronic device. For example, changes in an intensity of a face portion of inferred panel 380 of a rendered representation 391 (e.g., a video signal) over time may correspond to a current heart rate of the user as described with respect to FIG. 3. In some implementations, the video signal may include intensity based on live IR ECAM data depicting each eye and some surrounding areas captured while the user wears the wearable device (e.g., an HMD) as described with respect to FIG. 3. Likewise, the video signal may include color data from a prior enrollment captured by an outward facing RGM camera on the HMD. In some implementations, the face portion may comprise a region of skin (e.g., depiction 240 of skin as described with respect to FIG. 2) in an eye region within an eye-box of the wearable device.

At block 706, the method 700 embeds data (e.g., a numerical code) into the video signal by altering the attribute of the face portion in the video signal over time such that the changes in the attribute of the face portion in the video signal over time correspond to both the current heart rate and the data. For example, as described with respect to FIG. 3, data 393 such as a numerical code may be embedded into a rendered representation 391 by altering an attribute of a face portion in the rendered representation 391 over time such that the changes in the attribute of the face portion in the rendered representation 391 over time correspond to the current heart rate and the data 393.

In some implementations, the current heartrate may be determined based on remote photoplethysmography (rPPG).

In some implementations, the current heartrate may be determined by: (a) extracting an average intensity over the face portion in the video signal to produce a raw signal; (b) filtering the raw signal to produce a filtered signal; (c) transforming the raw signal into a frequency domain to produce a transformed signal; and (d) determining the current heartrate based on the transformed signal as described with respect to FIG. 2.

In some implementations, embedding the data may include altering the face portion such that the transformed signals are added as additional peaks corresponding to data values as described with respect to FIG. 2. In some implementations, the additional peaks may have height values corresponding to discrete data values and the heigh values of the additional peaks may be dependent upon a height of a peak corresponding to the heartrate.

In some implementations additional data from a third device (e.g., device 127 or 129 of FIG. 1) may be used to identify a heartrate of the user and the heartrate of the user identified from the additional data from the third device may be used to confirm an identity of the user.

At block 708, the method 700 presents the video signal depicting the current appearance of the face portion on an outward-facing display of the wearable electronic device. For example, a rendered representation 391 depicting a current appearance of a face portion may be presented as a rendered representation on a 3D display 395 such as an outward-facing display of a wearable electronic device as described with respect to FIG. 3.

In some implementations, a reading device (e.g., device 123 of FIG. 1) may capture images of the user wearing the wearable electronic device (e.g., user 110 wearing device 120 in FIG. 1) and determine the data based on the images. In some implementations, the reading device may use uses remote rPPG to identify a heartrate in the captured images and use the heartrate to determine the data based on the images. In some implementations, the reading device may: (a) capture images of the user wearing the wearable electronic device; (b) identify a first patch of skin of the user (patch 508 of skin of user 504 as described with respect to FIG. 5) wearing the wearable electronic device directly visible in the images; (c) identify a second patch of skin of the user (patch 510 of skin of user 504 as described with respect to FIG. 5) wearing the wearable electronic device in the video signal displayed on the outward-facing display of the wearable electronic device; (d) compare heartrates identified from the first patch of skin and the second patch of skin; and (e) decode the data and/or authenticate the user based on comparing the heartrates.

In some implementations, embedding the data into the video signal presented on the outward-facing display of the wearable electronic device may enable another device (e.g., device 123 or 127 of FIG. 1) to automatically connect or authenticate to share content with the wearable electronic device.

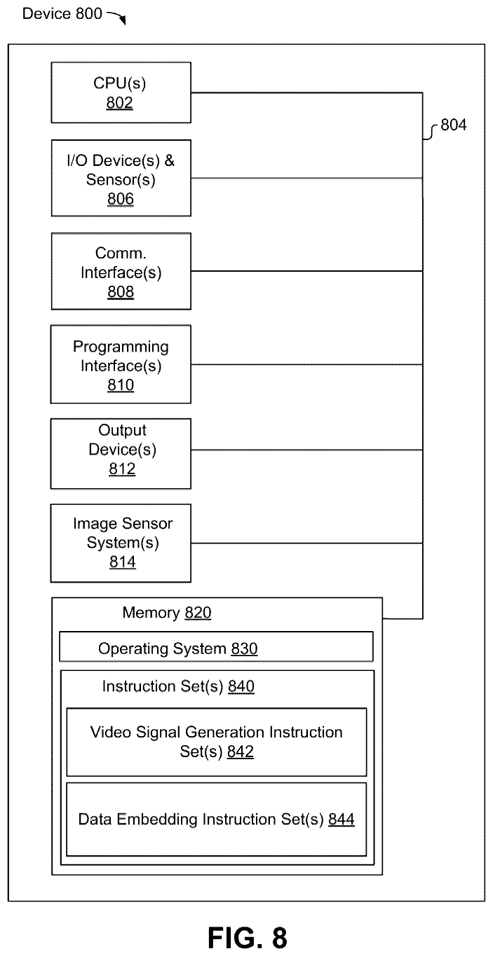

FIG. 8 is a block diagram of an example device 800. Device 800 illustrates an exemplary device configuration for electronic devices 120, 123, 127 and 129 of FIG. 1. While certain specific features are illustrated, those skilled in the art will appreciate from the present disclosure that various other features have not been illustrated for the sake of brevity, and so as not to obscure more pertinent aspects of the implementations disclosed herein. To that end, as a non-limiting example, in some implementations the device 800 includes one or more processing units 802 (e.g., microprocessors, ASICs, FPGAs, GPUs, CPUs, processing cores, and/or the like), one or more input/output (I/O) devices and sensors 806, one or more communication interfaces 808 (e.g., USB, FIREWIRE, THUNDERBOLT, IEEE 802.3x, IEEE 802.11x, IEEE 802.14x, GSM, CDMA, TDMA, GPS, IR, BLUETOOTH, ZIGBEE, SPI, I2C, and/or the like type interface), one or more programming (e.g., I/O) interfaces 810, output devices (e.g., one or more displays) 812, one or more interior and/or exterior facing image sensor systems 814, a memory 820, and one or more communication buses 804 for interconnecting these and various other components.

In some implementations, the one or more communication buses 804 include circuitry that interconnects and controls communications between system components. In some implementations, the one or more I/O devices and sensors 806 include at least one of an inertial measurement unit (IMU), an accelerometer, a magnetometer, a gyroscope, a thermometer, one or more physiological sensors (e.g., blood pressure monitor, heart rate monitor, blood oxygen sensor, blood glucose sensor, etc.), one or more microphones, one or more speakers, a haptics engine, one or more depth sensors (e.g., a structured light, a time-of-flight, or the like), one or more cameras (e.g., inward facing cameras and outward facing cameras of an HMD), one or more infrared sensors, one or more heat map sensors, and/or the like.

In some implementations, the one or more displays 812 are configured to present a view of a physical environment, a graphical environment, an extended reality environment, etc. to the user. In some implementations, the one or more displays 812 are configured to present content (determined based on a determined user/object location of the user within the physical environment) to the user. In some implementations, the one or more displays 812 correspond to holographic, digital light processing (DLP), liquid-crystal display (LCD), liquid-crystal on silicon (LCoS), organic light-emitting field-effect transitory (OLET), organic light-emitting diode (OLED), surface-conduction electron-emitter display (SED), field-emission display (FED), quantum-dot light-emitting diode (QD-LED), micro-electromechanical system (MEMS), and/or the like display types. In some implementations, the one or more displays 812 correspond to diffractive, reflective, polarized, holographic, etc. waveguide displays. In one example, the device 800 includes a single display. In another example, the device 800 includes a display for each eye of the user.

In some implementations, the one or more image sensor systems 814 are configured to obtain image data that corresponds to at least a portion of the physical environment 100. For example, the one or more image sensor systems 814 include one or more RGB cameras (e.g., with a complimentary metal-oxide-semiconductor (CMOS) image sensor or a charge-coupled device (CCD) image sensor), monochrome cameras, IR cameras, depth cameras, event-based cameras, and/or the like. In various implementations, the one or more image sensor systems 814 further include illumination sources that emit light, such as a flash. In various implementations, the one or more image sensor systems 814 further include an on-camera image signal processor (ISP) configured to execute a plurality of processing operations on the image data.

In some implementations, sensor data may be obtained by device(s) (e.g., devices 105 and 110 of FIG. 1) during a scan of a room of a physical environment. The sensor data may include a 3D point cloud and a sequence of 2D images corresponding to captured views of the room during the scan of the room. In some implementations, the sensor data includes image data (e.g., from an RGB camera), depth data (e.g., a depth image from a depth camera), ambient light sensor data (e.g., from an ambient light sensor), and/or motion data from one or more motion sensors (e.g., accelerometers, gyroscopes, IMU, etc.). In some implementations, the sensor data includes visual inertial odometry (VIO) data determined based on image data. The 3D point cloud may provide semantic information about one or more elements of the room. The 3D point cloud may provide information about the positions and appearance of surface portions within the physical environment. In some implementations, the 3D point cloud is obtained over time, e.g., during a scan of the room, and the 3D point cloud may be updated, and updated versions of the 3D point cloud obtained over time. For example, a 3D representation may be obtained (and analyzed/processed) as it is updated/adjusted over time (e.g., as the user scans a room).

In some implementations, sensor data may be positioning information, some implementations include a VIO to determine equivalent odometry information using sequential camera images (e.g., light intensity image data) and motion data (e.g., acquired from the IMU/motion sensor) to estimate the distance traveled. Alternatively, some implementations of the present disclosure may include a simultaneous localization and mapping (SLAM) system (e.g., position sensors). The SLAM system may include a multidimensional (e.g., 3D) laser scanning and range-measuring system that is GPS independent and that provides real-time simultaneous location and mapping. The SLAM system may generate and manage data for a very accurate point cloud that results from reflections of laser scanning from objects in an environment.

Movements of any of the points in the point cloud are accurately tracked over time, so that the SLAM system can maintain precise understanding of its location and orientation as it travels through an environment, using the points in the point cloud as reference points for the location.

In some implementations, the device 800 includes an eye tracking system for detecting eye position and eye movements (e.g., eye gaze detection). For example, an eye tracking system may include one or more infrared (IR) light-emitting diodes (LEDs), an eye tracking camera (e.g., near-IR (NIR) camera), and an illumination source (e.g., an NIR light source) that emits light (e.g., NIR light) towards the eyes of the user. Moreover, the illumination source of the device 800 may emit NIR light to illuminate the eyes of the user and the NIR camera may capture images of the eyes of the user. In some implementations, images captured by the eye tracking system may be analyzed to detect position and movements of the eyes of the user, or to detect other information about the eyes such as pupil dilation or pupil diameter. Moreover, the point of gaze estimated from the eye tracking images may enable gaze-based interaction with content shown on the near-eye display of the device 800.

The memory 820 includes high-speed random-access memory, such as DRAM, SRAM, DDR RAM, or other random-access solid-state memory devices. In some implementations, the memory 820 includes non-volatile memory, such as one or more magnetic disk storage devices, optical disk storage devices, flash memory devices, or other non-volatile solid-state storage devices. The memory 820 optionally includes one or more storage devices remotely located from the one or more processing units 802. The memory 820 includes a non-transitory computer readable storage medium.

In some implementations, the memory 820 or the non-transitory computer readable storage medium of the memory 820 stores an optional operating system 830 and one or more instruction set(s) 840. The operating system 830 includes procedures for handling various basic system services and for performing hardware dependent tasks. In some implementations, the instruction set(s) 840 include executable software defined by binary information stored in the form of electrical charge. In some implementations, the instruction set(s) 840 are software that is executable by the one or more processing units 802 to carry out one or more of the techniques described herein.

The instruction set(s) 840 includes a video signal generation instruction set 842 and a data embedding instruction set 844. The instruction set(s) 840 may be embodied as a single software executable or multiple software executables.

The video signal generation instruction set 842 is configured with instructions executable by a processor to generate a video signal depicting a current appearance of a face portion of a user such that changes in an attribute (e.g., intensity) of the face portion in the video signal over time correspond to a current heart rate of a user wearing a wearable electronic device.

The enhanced display instruction set 844 is configured with instructions executable by a processor to embed data into a video signal by altering attribute of a face portion in the video signal over time such that the changes in the attribute of the face portion in the video signal over time correspond to both a current heart rate and the data.

Although the instruction set(s) 840 are shown as residing on a single device, it should be understood that in other implementations, any combination of the elements may be located in separate computing devices. Moreover, FIG. 8 is intended more as functional description of the various features which are present in a particular implementation as opposed to a structural schematic of the implementations described herein. As recognized by those of ordinary skill in the art, items shown separately could be combined and some items could be separated. The actual number of instructions sets and how features are allocated among them may vary from one implementation to another and may depend in part on the particular combination of hardware, software, and/or firmware chosen for a particular implementation.

Those of ordinary skill in the art will appreciate that well-known systems, methods, components, devices, and circuits have not been described in exhaustive detail so as not to obscure more pertinent aspects of the example implementations described herein. Moreover, other effective aspects and/or variants do not include all of the specific details described herein. Thus, several details are described in order to provide a thorough understanding of the example aspects as shown in the drawings. Moreover, the drawings merely show some example embodiments of the present disclosure and are therefore not to be considered limiting.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, e.g., one or more modules of computer program instructions, encoded on computer storage medium for execution by, or to control the operation of, data processing apparatus. Alternatively, or additionally, the program instructions can be encoded on an artificially generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. Unless specifically stated otherwise, it is appreciated that throughout this specification discussions utilizing the terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general purpose computing apparatus to a specialized computing apparatus implementing one or more implementations of the present subject matter. Any suitable programming, scripting, or other type of language or combinations of languages may be used to implement the teachings contained herein in software to be used in programming or configuring a computing device.

Implementations of the methods disclosed herein may be performed in the operation of such computing devices. The order of the blocks presented in the examples above can be varied for example, blocks can be re-ordered, combined, and/or broken into sub-blocks. Certain blocks or processes can be performed in parallel. The operations described in this specification can be implemented as operations performed by a data processing apparatus on data stored on one or more computer-readable storage devices or received from other sources.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various elements, these elements should not be limited by these terms. These terms are only used to distinguish one element from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

What is claimed is:

1. A method comprising:
at a wearable electronic device having a processor:
generating a video signal depicting a current appearance of a face portion, the video signal generated based on sensor data captured via one or more sensors, wherein changes in an attribute of the face portion in the video signal over time correspond to a current heart rate of a user wearing the wearable electronic device;
embedding data into the video signal by altering the attribute of the face portion in the video signal over time such that the changes in the attribute of the face portion in the video signal over time correspond to both the current heart rate and the data; and
presenting the video signal depicting the current appearance of the face portion on an outward-facing display of the wearable electronic device.

2. The method of claim 1, wherein the data is a numerical code.

3. The method of claim 1, wherein the wearable electronic device is a head mounted device (HMD) and the face portion is a region of skin in an eye region within an eye-box of the HMD.

4. The method of claim 1 further comprising:
determining the current heartrate based on remote photoplethysmography (rPPG).

5. The method of claim 4, wherein determining the current heartrate comprises:
extracting an average intensity over the face portion in the video signal to produce a raw signal;
filtering the raw signal to produce a filtered signal;
transforming the raw signal into a frequency domain to produce a transformed signal; and
determining the current heartrate based on the transformed signal.

6. The method of claim 5, wherein embedding the data comprises altering the face portion such that the transformed signals are added as additional peaks corresponding to data values.

7. The method of claim 6, wherein the additional peaks have height values corresponding to discrete data values.

8. The method of claim 5, wherein height values of the additional peaks are dependent upon a height of a peak corresponding to the heartrate.

9. The method of claim 1, wherein a reading device captures images of the user wearing the wearable electronic device and determines the data based on the images.

10. The method of claim 9, wherein the reading device uses remote photoplethysmography (rPPG) to identify a heartrate in the captured images and uses the heartrate to determine the data based on the images.

11. The method of claim 1, wherein a reading device:
captures images of the user wearing the wearable electronic device;
identifies a first patch of skin of the user wearing the wearable electronic device directly visible in the images;
identifies a second patch of skin of the user wearing the wearable electronic device in the video signal displayed on the outward-facing display of the wearable electronic device;
compares heartrates identified from the first patch and the second patch; and
decodes the data based on comparing the heartrates.

12. The method of claim 1, wherein a reading device:
captures images of the user wearing the wearable electronic device;
identifies a first patch of skin of the user wearing the wearable electronic device directly visible in the images;
identifies a second patch of skin of the user wearing the wearable electronic device in the video signal displayed on the outward-facing display of the wearable electronic device;
compares heartrates identified from the first patch and the second patch; and
authenticates the user based on comparing the heartrates.

13. The method of claim 1 further comprising using additional data from a third device to identify a heartrate of the user.

14. The method of claim 13, wherein the heartrate of the user identified from the additional data from the third device is used to confirm an identity of the user.

15. The method of claim 1, wherein embedding the data into the video signal presented on the outward-facing display of the wearable electronic device enables another device to automatically connect or authenticate to share content with the wearable electronic device.

16. The method of claim 1, wherein the electronic device is a head-mounted device (HMD) displaying the video signal to present a view of an eye region of the user.

17. A wearable device comprising:
a non-transitory computer-readable storage medium;
one or more sensors; and
one or more processors coupled to the non-transitory computer-readable storage medium, wherein the non-transitory computer-readable storage medium comprises program instructions that, when executed on the one or more processors, cause the one or more processors to perform operations comprising:
generating a video signal depicting a current appearance of a face portion, the video signal generated based on sensor data captured via the one or more sensors, wherein changes in an attribute of the face portion in the video signal over time correspond to a current heart rate of a user wearing the wearable electronic device;
embedding data into the video signal by altering the attribute of the face portion in the video signal over time such that the changes in the attribute of the face portion in the video signal over time correspond to both the current heart rate and the data; and
presenting the video signal depicting the current appearance of the face portion on an outward-facing display of the wearable electronic device.

18. The wearable device of claim 17, wherein the data is a numerical code.

19. The wearable device of claim 17, wherein the wearable electronic device is a head mounted device (HMD) and the face portion is a region of skin in an eye region within an eye-box of the HMD.

20. A non-transitory computer-readable storage medium, storing program instructions executable on a device including one or more processors to perform operations comprising:
generating a video signal depicting a current appearance of a face portion, the video signal generated based on sensor data captured via one or more sensors, wherein changes in an attribute of the face portion in the video signal over time correspond to a current heart rate of a user wearing the wearable electronic device;
embedding data into the video signal by altering the attribute of the face portion in the video signal over time such that the changes in the attribute of the face portion in the video signal over time correspond to both the current heart rate and the data; and
presenting the video signal depicting the current appearance of the face portion on an outward-facing display of the wearable electronic device.
